(19) United States
(12) Patent Application Publication
(10) Pub. No.: US 2024/0382871 A1
(43) Pub. Date: Nov. 21, 2024

(54) IONIC LIQUID DEMULSIFIERS FOR EMULSION SEPARATION AND BITUMEN DEWATERING

(71) Applicant: THE GOVERNORS OF THE UNIVERSITY OF ALBERTA, Edmonton (CA)

(72) Inventors: Hongbo Zeng, Edmonton (CA); Mostafa Khodakarami, Edmonton (CA)

(21) Appl. No.: 18/737,311

(22) Filed: Jun. 7, 2024

Related U.S. Application Data
(60) Provisional application No. 63/503,320, filed on May 19, 2023.

(51) Int. Cl. B01D 17/04; C07C 53/134; C07C 211/63

(52) U.S. Cl. CPC B01D 17/047; C07C 53/134; C07C 211/63; C07C 2601/08; C07C 2601/14

(57) ABSTRACT

Embodiments disclosed herein generally relate to ionic liquid demulsifiers for separation of water from water-in-oil emulsions. The ionic liquid demulsifiers may formed of an amphiphilic quaternary ammonium cation with general formula of R₁R₂R₃N+CH₃ (R₁, R₂, and R₃ are alkyl chains with 6 to 12 carbon atoms) and an amphiphilic anion based on carboxylic acids. Anionic component can be based on linear unsaturated or saturated fatty acids with 8 to 18 carbon atoms, and cyclo-carboxylic acids containing cyclohexane, cyclopentane and naphthenic rings. The length of the alkyl chains and number of carbon atoms in the anionic components, as well as the operational parameters of demulsification process may be dependent on the water content and the nature of stabilizing species of the emulsions.

### CROSS-REFERENCE TO RELATED APPLICATION

The present application claims priority to U.S. provisional patent application No. 63/503,320, filed on May 19, 2023, the entire contents which are incorporated herein by reference.

### FIELD

The present invention generally relates to chemical compositions and formulations to resolve oil and water emulsions and in particular water-in-oil emulsions. More particularly, embodiments herein relate to synthesis and use of ionic liquid demulsifiers for removal of water from water-in-oil emulsions, including water-in-oil emulsions encountered in bitumen or heavy oil production and oil sand treatment processes.

### BACKGROUND

Formation of emulsions, comprising oil and water, commonly occur in the extraction, production, and processing/refining of conventional oil, as well as recovering bitumen in oil sand treatment process. In these applications, often oil is the continuous phase and water is the dispersed phase, forming a very stable water-in-oil emulsion. The stability of these emulsions is believed to be due to the presence of natural surface-active components in heavy oil, which form an interfacial layer around the water droplets and hinder their coalescence. Asphaltenes, resins, naphthenic acids, phenols, and waxes are the main polyaromatic compounds in crude oil that can contribute to the stability of water-in-oil emulsions, along with inorganic species such as ions (e.g., Na+, Ca2+, Mg2+, K+) and fine solid particles (e.g., clays). It is desirable to break these emulsions as they can adversely impact the oil/bitumen recovery, final product quality, and can also cause serious operational problems such as fouling in transportation pipelines and corrosion in downstream refinery facilities.

To that end, demulsifiers are used to break these emulsions. The efficiency of demulsifiers often depends on the properties of the oil/water interface in an emulsion, as well as the bulk emulsion properties.

Generally, it is believed that demulsifiers should have higher interfacial activity compared to the surface-active species present in oil. In this manner, they can be used to change the mechanical properties of stabilizing interfacial layers, as well as disrupting the stabilizing interfacial layers to provide favorable zones for coalescence between water droplets.

Due to the complex nature of bulk and interfacial phases, development of highly efficient and cost-effective demulsifiers is challenging and a demanding process. A need therefore exists for demulsifiers with an improved performance to efficiently break the emulsion in a relatively short time and low concentrations.

### SUMMARY OF VARIOUS EMBODIMENTS

Embodiments herein relate to surface-active compounds for the demulsification of stabilized water-in-oil emulsions, which can be used for removal of water from bitumen in oil sand treatment process. In some examples, the water-in-oil emulsions—which are being demulsified—are stabilized by complex polyaromatic compounds (e.g., asphaltene), salt ions, and/or fine solid particles.

Generally, the invention comprises use and synthesis of an ionic liquid demulsifier comprising: (i) an amphiphilic quaternary ammonium cation with aliphatic hydrophobic tails, having a general formula of R₁R₂R₃N+CH₃, wherein R₁, R₂, and R₃ are each alkyl chains with 6 to 12 carbon atoms, and (ii) an amphiphilic anion comprising a carboxylic acid with a cyclic or linear hydrophobic tail.

In some examples, the anionic carboxylic acid can be based on linear or branched, unsaturated or saturated fatty acids with 8 to 18 carbon atoms, or cyclo-carboxylic acids containing cyclohexane, cyclopentane or naphthenic rings. The length of the alkyl chains and number of carbon atoms in the anionic component, of the ionic liquids, as well as the operational parameters of demulsification process may be dependent on the water content and the nature of the stabilizing species of the emulsions.

The disclosure further relates to a process for demulsification of heavy oil or bitumen by treating them with the disclosed ionic liquid demuslifier. In at least one example, heavy oil or bitumen are treated with the ionic liquid demulsifier, applied in the range of 25-300 ppm, and in the temperature range of 40-80° C. The demulsifier can be applied to the emulsion by shaking to obtain demulsified oil or bitumen with <1% water. To that end, the synthesized ionic liquids are readily soluble in variety of solvents including aromatic and aliphatic hydrocarbons as well as naphtha, as a mixture of volatile liquid hydrocarbons used in oil sand treatment process.

In accordance with this disclosure, and in at least one broad aspect, there is provided a ionic liquid demulsifier comprising an amphiphilic quaternary ammonium cation with the general formula of R₁R₂R₃N⁺ R₅ (wherein R₁, R₂, and R₃ are alkyl chains with 6 to 12 carbon atoms, and R₅ is methyl, ethyl or propyl), and an amphiphilic anion comprising a carboxylic acid.

In another broad aspect, the ionic liquid demulsifier is synthesized by converting a quaternary ammonium halide salt to hydroxide form, then the hydroxide anion of the quaternary ammonium reacts with the hydrogen of the carboxyl group, yielding the target ionic liquid demulsifier and water byproduct.

In some embodiments, the anionic component comprises a fatty acid, saturated or unsaturated, having 8 to 18 carbon atoms.

In some embodiments, the anionic component is a cyclo-carboxylate obtained from 4-cyclohexylbutric acid.

In some embodiments, the anionic component is a cyclo-carboxylate obtained from cyclohexyl acetic acid.

In some embodiments, the anionic component is a cyclo-carboxylate obtained from naphthenic acid.

In still another broad aspect, there is provided a process of demulsification of water-in-oil emulsions, wherein the above-noted demulsifier compositions are brought into contact and mixed with the emulsion to act upon it in any method generally known in the art of oil production and oil sand treatment industries for removing water or breaking the emulsion using a chemical reagent.

In some embodiments, the process of demulsification involves the above-noted demulsifier compositions being dissolved in various organic solvents including aliphatic hydrocarbons, aromatic solvents, and naphtha, as a mixture of volatile liquid hydrocarbons widely used in oil sand treatment process.

In some embodiments, the oil phase can be light oil, heavy oil, and bitumen froth of oil sand treatment process.

In some embodiments, the above-noted demulsifier compositions can be used in the concentration range of 25 to 4000 ppm and the temperature range of 20° C. to 80° C.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 is a schematic of an example process for preparing and synthesizing an ionic liquid demulsifier, in accordance with embodiments disclosed herein.

Further aspects and features of the example embodiments described herein will appear from the following description taken together with the accompanying drawings.

### DETAILED DESCRIPTION OF THE EMBODIMENTS

Embodiments described herein generally relate to ionic liquid demulsifiers for removal of water, from water-in-oil emulsions. The disclosed demulsifiers can be used in low dosages for removing water from water-in-bitumen emulsions, such as those encountered in oil sand treatment process. It is believed that the disclosed demulsifier are composed of more environmentally acceptable constituents than conventional demulsifiers.

### I. DEFINITIONS

Any term or expression not expressly defined herein shall have its commonly accepted definition understood by a person skilled in the art. As used herein, the following terms have the following meanings.

“Liquid emulsion” (or simply “emulsion”, as used herein) refers to a mixture of at least two immisicble liquids where one liquid is in a fine dispersion of droplets (e.g., the dispersed phase) within another liquid, which is the continuous phase. Processes involving single phase or multiphase emulsions are within the scope of the present invention.

“Water-in-oil emulsion” is an emulsion wherein water is dispersed in a continuous oil phase.

“Demulsifiers” refer to a class of specialty chemicals used to separate emulsions.

“Heavy oil” refers to oil having an asphaltic, dense, viscous nature, and high asphaltene content. Although variously defined, the upper limit for heavy oil is 22° API gravity with a viscosity of 100 cp (centipoise). “Bitumen” is extra heavy oil that has even higher viscosity and density (an API gravity of between about 7° and 14°) and a higher content of asphaltenes. Heavy oil may comprise bitumen recovered from natural unconventional oil sand deposits, such as the Athabasca oil sands in Alberta, Canada.

### II. EXAMPLE STRUCTURE FOR DEMULSIFIER

In general, the disclosed ionic liquid demulsifiers are based on linear and cyclo-carboxylic acids. In one embodiment, an ionic liquid demulsifier comprises: (i) an amphiphilic ammonium-based cation with aliphatic hydrophobic tails and (ii) an amphiphilic carboxylate anion having a cyclic or linear hydrophobic tail. The ammonium-based cation may comprise a quaternary ammonium cation with the general formula of R₁R₂R₃N+ R₅

In some embodiments, a demulsifier may have the structure of Formula I:

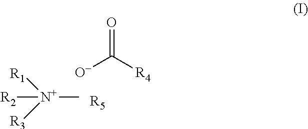

where R₁, R₂, and R₃ are branched or linear alkyl chains, independent from each other, wherein each alkyl chain comprises between 6 to 12 carbon atoms; R₄ is a saturated or unsaturated, linear, branched or cyclic moiety having between 7 to 17 carbon atoms; and R₅ is methyl, ethyl or propyl or isopropyl. In preferred embodiments, R₄ includes an aliphatic or aromatic ring, which may be, for example, a cyclohexane or cyclopentane.

In preferred embodiments, the anionic carboxylic acid may octanoic acid [C8], decanoic acid [C10], or dodecanoic acid [C12]. In these embodiments, R₄ is a linear chain of 7, 9 or 11 atoms respectively.

In one embodiment, R₄ comprises a cyclic structure, such as a cyclohexane of Formula II:

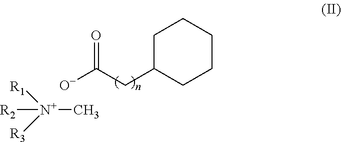

where R₁, R₂, and R₃ are defined as above; and n is 1 to 9.

In another embodiment, R₄ comprises a cyclopentane, such as that of Formula III:

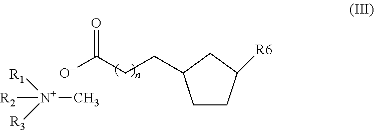

Wherein R₁, R₂, and R₃ are defined as above; and R₆ is hydrogen, carbon or a linear or branched alkyl chain having between 2 and 13 carbon atoms; and n is between 0 and 12. Preferably, R₆ is methyl, ethyl, propyl or isopropyl. Preferably, the total number of carbon atoms of R₄ is less than or equal to 17.

### III. EXAMPLE PROCESS FOR PREPARING AND SYNTHESIZING DEMULSIFIER

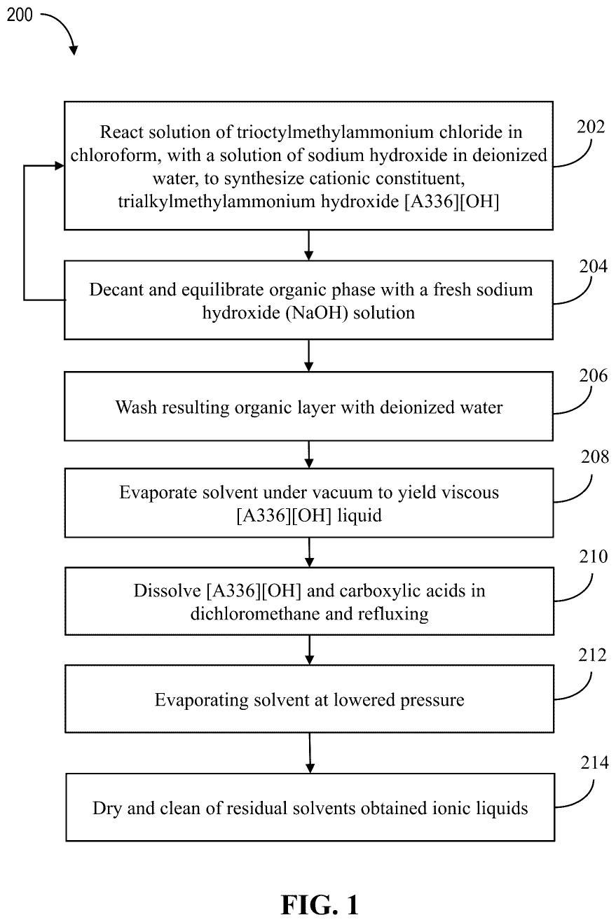

FIG. 1 shows an example process (200) for preparing and synthesizing ionic liquid demulsifiers, in accordance with disclosed embodiments.

As shown, at step (202), the cationic constituent of the demulsifier—trialkylmethylammonium hydroxide—is synthesized by reacting a 0.5 mole (M) solution of trioctylmethylammonium chloride ([Aliquat 336 (A336)][Cl])

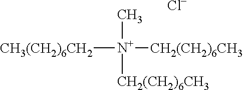

in chloroform with a 5 M solution of sodium hydroxide in deionized water. This reaction may be carried out for one hour.

At step (204), after the reaction is complete, the organic phase is decanted and equilibrated with a fresh 5 M sodium hydroxide (NaOH) solution.

Steps (202) and (204) may be repeated, until the chloride content is negligible. In some cases, the chloride content is tested with an acidified silver nitrate solution. In at least one example, acts (202) and (204) are repeated eight to ten times, to achieve negligible chloride content.

At step (206), after the chloride content is determined to be negligible, the resulting organic layer is washed with deionized water. At (208), the solvent is then evaporated under vacuum to yield a viscous [A336][OH] liquid.

At step (210), the [A336][OH] and carboxylic acids are dissolved in dichloromethane (e.g., molar ratio 1:1) and refluxed for example, at 30° C. for 12 hours. Preferred

At step (212), viscous ionic liquids are obtained after evaporating the solvent in lowered pressure. At step (214), the obtained ionic liquids are dried and cleaned of residual solvents by thermal drying. In some examples, the thermal drying occurs at 70° C. in a vacuum oven for 7-8 hours.

### IV. DEMULSIFICATION OF WATER-IN-OIL EMULSIONS USING DEMULSIFIER

In the process of demulsification of water-in-oil emulsions, the ionic liquid demulsifier can be brought into contact, and mixed, with the emulsion to act upon it. In example applications involving oil production and oil sand treatment, the demulsifier can be brought into contact and mixed with the emulsion using any method known in the art of oil production and oil sand treatment for removing water or breaking the emulsion using a chemical reagent.

The ionic liquids can be dissolved in a suitable solvent to be added to the emulsion. For example, the disclosed ionic liquid demulsifiers can be readily dissolved in various organic solvents including aliphatic hydrocarbons, aromatic solvents, and naphtha, as a mixture of volatile liquid hydrocarbons which are widely used in oil sand treatment process.

The ionic liquid demulsifiers may also be used in combination with other emulsion breakers, viscosity reducers, corrosion inhibitors, and may be assisted with other demulsification techniques such as microwave, ultrasound, and electrical techniques.

Influential parameters—in the demulsification process—such as ionic liquid concentration, temperature, settling time, water content of the emulsion, and agitation may vary depending on the properties of the emulsion and actual system.

### V. EXPERIMENTAL TESTING OF DEMULSIFIER

Laboratory bottle tests on model emulsion or actual emulsion were conducted to determine the optimum operational condition and required formulation for treating the actual production system.

In a typical bottle test, the ionic liquid demulsifiers are introduced into the emulsion by injecting them beneath the top surface of the emulsion. The bottle is shaken or agitated to well disperse the demulsifier molecules in emulsion and then mixing is stopped and the emulsion is allowed to separate. The rate of water/oil separation is monitored and recorded, and the demulsification efficiency (DE) is calculated by Equation (1);

$$DE \% = \frac{{V}_{W}}{{V}_{0 , W}} \times 100 \qquad ( 1 )$$

where V_{w} is the volume of separated water, and V_{0,w} is the volume of water in emulsion before demulsification.

In at least one example, to prepare a model organic phase for laboratory bottle tests, C5-asphaltenes (asphaltenes obtained from bitumen by precipitation with pentane) were added to toluene and sonicated for 15 minutes for complete dissolution. Further, to prepare the model water-in-oil emulsion, a certain amount of deionized water was slowly added to the model organic phase while stirring by a homogenizer, e.g., at 18000 rpm for 30 min. The resulting solution was then allowed to settle (e.g., for 2 hours), then the settled emulsion phase is separated from the supernatant organic phase for demulsification tests. In at least one example, the demulsification of model water-in-oil emulsions were conducted at 60° C.

For water-in-bitumen demulsification experiments, an emulsion was prepared by mixing the bitumen froth with naphtha with the naphtha to bitumen (N/B) ratio of 0.42. This ratio is generally used in the industrial oil sand treatment process. The demulsification tests on water-in-bitumen emulsions were conducted at 80° C., which is the temperature usually used in actual industrial process. The water content of the top oil layer was measured by taking samples from ˜1 cm beneath the top surface and analyzing them with a Karl Fischer® titration technique.

### VI. EXAMPLE SPECIFIC EMBODIMENTS

The following examples illustrate specific aspects of the exemplary embodiments described above.

As noted previously, the anionic component of the liquid demulsifier can be based on linear unsaturated or saturated fatty acids with 8 to 18 carbon atoms, and cyclo-carboxylic acids containing cyclohexane, cyclopentane and naphthenic rings.

### (i) Example No. 1: Ionic Liquid Demulsifier With Three Linear Carboxylic Acids

An ionic liquid demulisifier with three linear carboxylic acids, also known as fatty acids, were used as representative examples for being prepared and employed for demulsification process.

To that end, three linear carboxylic acids were octanoic acid [C8], decanoic acid [C10], and dodecanoic acid [C12], respectively, were used for the synthesis of fatty acid-based ionic liquid demulsifiers.

In this example, the synthesis yields were approximately 96%, 95% and 96% for [A336][C8], [A336][C10], and [A336][C12], respectively. It was found that the viscosity of fatty acid-based ionic liquids increased as the length of the alkyl chain on carboxylate group increases. Viscosity of [A336][C8], [A336][C10], and [A336][C12] were found to be 1.1, 1.3, and 1.5 Pa.S (pascal-second), respectively.

The thermal decomposition of [A336][C8], [A336][C10], and [A336][C12] started at a temperature range of 160-180° C. As the length of the alkyl chain on carboxylate group increased, the decomposition of the fatty acid-based ionic liquids started at higher temperatures.

Ionic liquid dosages ranging from 50 to 4000 ppm were studied for model water-in-oil emulsion separation to evaluate the performance of these ionic liquids in different concentration ranges. The “ionic liquid dosage”, used herein, refers to the total concentration of ionic liquid in whole volume of emulsion.

For model emulsions containing 800 mg/l (milligrams per liter) of C5-asphaltenes and 20% deionized water, demulsification efficiency greater than 90% was achieved in five (5) minutes for fatty acid-based ionic liquids with concentrations higher than 1000 ppm (parts per million).

The effect of ionic liquid concentration on demulsification efficiency was found to be different for the ionic liquids with different lengths of alkyl chains of anionic linear carboxylate groups.

For concentrations lower than 1000 ppm, as the length of alkyl chain on carboxylate group increased, the performance of ionic liquids was improved in terms of demulsification kinetics. At ionic liquids concentrations above 1000 ppm, the demulsification kinetics of examined fatty acid-based ionic liquids with shorter carboxylate groups were slightly higher than longer ones.

Water content and demulsification efficiency of the fatty acid-based ionic liquids were appreciated to have an inverse relation with each other. As the water content decreased from 20% to 5%, the demulsification efficiency of 300 ppm of ionic liquids with linear carboxylate groups decreased from ˜99% to ˜85% after giving two (2) hours of settling time. As the water content of the emulsion decreased, it gave more time favors the demulsification efficiency.

The optimum temperature of demulsification for the fatty acid-based ionic liquids was found to be in a range between 60° C. to 80° C. The achieved results indicated that these ionic liquids were suitable for demulsification of actual water-in-bitumen emulsion, which is treated at approximately 80° C. in the industrial process.

At studied ionic liquid concentration (e.g., 300 ppm), the efficiency of ionic liquids with longer alkyl chains on carboxylate group was found to be higher at all temperatures. Decreasing the temperature from 60° C. to 40° C. resulted in decrease of demulsification efficiency of the examined fatty acid-based ionic liquids from approximately 99% to 80-85%, after giving two (2) hours of settling time with 300 ppm of ionic liquids.

Additionally, water salinity was found to lead to higher emulsion stability and lower water separation kinetics and efficiency in the case of model water-in-oil demulsification process.

Quality of separated water was further improved when the concentration of fatty acid-based ionic liquids used for demulsification process was decreased. As the concentration of ionic liquids in emulsion decreased from 1000 ppm to 300 ppm, the turbidity of separated water decreased from 4.4 NTU (Nephelometric Turbidity Units) to 3.3 NTU for [A336][C8], from 2.4 NTU to 2 NTU for [A336][C10], and from 2.1 NTU to 1.5 NTU for [A336][C12].

The interfacial tension between toluene and deionized water was found to be approximately 32 mN/m (millinewtons/metre). When the toluene phase contains 300 ppm of [A336][C8], [A336][C10], and [A336][C12], the interfacial tension decreased to 9.6, 9.9, 10.3 mN/m, respectively. Increasing the concentration of ionic liquids in toluene lead to an increase in the interfacial tension. Increasing the length of alkyl chain on carboxylate group resulted in higher interfacial tension. The presence of salt ions in water phase resulted in lower interfacial tension between water phase and toluene phase containing ionic liquids. The presence of salt ions in water lowered the influence of alkyl chain length of the ionic liquids on the interfacial tension between water and toluene phase containing ionic liquids.

Initial water content of water-in-bitumen emulsion was appreciated to be an influential parameter affecting the demulsification efficiency of fatty acid-based ionic liquids. For the water-in-bitumen emulsion containing ˜20% water, the demulsification efficiency reached ˜90-95%, using 100 ppm of fatty acid-based ionic liquids and after three (3) hours of settling time. Increasing the settling time to 4-5 hours can increase the demulsification efficiency to ˜98%. Increasing the length of alkyl chain had a positive effect on the demulsification efficiency.

For a water-in-bitumen emulsion with initial water content of ˜11%, the demulsification efficiency of 100 ppm of tested fatty acid-based ionic liquids reaches ˜60-65% after three (3) hours of settling time. Decreasing the concentration of ionic liquids to 50 ppm increases the demulsification efficiency to 70-90% after 4-5 hours of settling time.

For a water-in-bitumen emulsion with initial water content of ˜5-6%, the demulsification efficiency of 100 ppm of tested fatty acid-based ionic liquids reaches ˜53-60% after 3 hours of settling time and it increases to ˜75-80% after 5 hours of settling time. It took ˜6 hours to reach water content <1% in the top oil phase.

Concentration of ionic liquid demulsifiers also has a significant effect on the water-in-bitumen demulsification efficiency. Decreasing the concentration of tested ionic liquids from 1000 ppm to 300 ppm resulted in an increase of demulsification efficiency from 70-75% to 80-87% after approximately 3 hours, when initial water content is ˜20%.

The naphtha to bitumen (N/B) ratio was found to have a significant effect on the demulsification efficiency of tested ionic liquids. Increasing the N/B ratio from 0.42 to 2 can increase the demulsification efficiency of 100 ppm of examined fatty acid-based ionic liquids to >99% in ˜2 hours and decrease the water content of top oil phase to <0.1% in ˜2 hours.

### (ii) Example No. 2: Synthesizing Using 4-Cyclohexylbutric Acid

In at least one example, 4-cyclohexylbutric acid, as a cyclo-carboxylic acid with chemical formula of C₁₀H₁₇O₂, is used to prepare an ionic liquid demulsifier based on a cyclo-carboxylic acid.

In this example, with reference to process (200) of FIG. 1—first, at step (202), trioctylmethylammonium hydroxide, [A336][OH], is synthesized by reacting a 0.5 M solution of trioctylmethylammonium chloride, [A336][Cl], in chloroform with a 5 M solution of sodium hydroxide in deionized water. The reaction can be carried out for one hour.

At step (204), the organic phase is decanted and equilibrated with a fresh 5 M NaOH solution. This procedure can be repeated for eight times and the chloride content being tested, for example, with an acidified silver nitrate solution to assure that the chloride content is negligible. The resulting organic layer is then washed with deionized water (step (206)), and the solvent is evaporated under vacuum to yield a viscous [A336][OH] liquid (step (208)).

Importantly, at step (210), the [A336][OH] and 4-cyclohexylbutric acid are dissolved in dichloromethane (molar ratio 1:1) and refluxed at 30° C. for 12-14 hours. The solvent is then evaporated (step (212)) using, for example, a rotary evaporator. This is then followed at step (214), by thermal drying at 70° C. in a vacuum oven for 7-8 hour to yield trioctylmethylammonium 4-cyclohexylbutrate, [A336][CHB]. The synthesis yield was ≈95%.

In this example, the viscosity of trioctylmethylammonium 4-cyclohexylbutrate, [A336][CHB], at 23° C. was found to be 1.8 Pa.S. The viscosity of [A336][CHB] was higher than its analogous fatty acid-based ionic liquid, [A336][C10].

It was also found that thermal decomposition of [A336][CHB] started at ˜175° C. The thermal stability of its analogous fatty acid-based ionic liquid, [A336][C10], was slightly higher. Full decomposition of [A336][CHB] occurred at 247° C., while leaving a little residue on the thermogravimetric analysis pan.

Ionic liquid dosages ranging from 50 to 4000 ppm were studied for model water-in-oil emulsion separation to determine the optimum [A336][CHB] concentration range for the actual demulsification process. The ionic liquid dosage stated hereafter is the total concentration of ionic liquid in whole volume of emulsion.

For model emulsion containing 800 mg/l of C5-asphaltenes and 20% deionized water, demulsification efficiency greater than 98% was found to be achieved in 20 minutes and 30 minutes by using 100 ppm and 50 of [A336][CHB], respectively. Demulsification kinetics was higher for higher ionic liquid dosages, however, ionic liquid dosages lower than 600 ppm worked better in longer times (>20 minutes) in terms of water separation percentage.

Regarding the effect of ionic liquid concentration, two separate regimes were observable. When the concentration of [A336][CHB] was higher than 1000 ppm, increasing the dosage of ionic liquid favored the demulsification efficiency, while at concentrations lower than 1000 ppm, decreasing the ionic liquid dosage favored the demulsification efficiency.

Water content and demulsification efficiency of [A336][CHB] appeared to have an inverse relation. As the water content decreased from 20% to 5%, the demulsification efficiency of 300 ppm of [A336][CHB] decreased from 97% to 85%, after giving 2 hours of settling time.

As the water content of the emulsion decreased, more time (>2 hours) was needed to reach water separation >95%. While water separation occurred immediately (5 minutes) when water content of the emulsion was 20%, it took about 30 minutes to see a considerable water separation when water content of the emulsion is 5%.

The optimum temperature of demulsification for [A336][CHB] was found to be in a range of 60° C. to 80° C. The achieved results indicated that [A336][CHB] could be potentially used for demulsification of actual water-in-bitumen emulsion which is treated at ˜80° C. in industrial process.

The demulsification kinetics of [A336][CHB] was also found to be higher than its analogous fatty acid-based ionic liquid, [A336][C10], at temperatures lower than 60° C.

Water salinity was also found to lead to higher emulsion stability and lower water separation kinetics and efficiency in the case of model water-in-oil demulsification process. In the presence of salt (NaCl) ions in the emulsion, the water separation efficiency of [A336][CHB] was higher than its analogous fatty acid-based ionic liquid, [A336][C10]. In the presence of NaCl ions, the demulsification efficiency of 300 ppm [A336][CHB] reached its maximum value of 94% in the first 20 minutes and then gradually decreased to ˜80% in 120 minutes.

The turbidity of separated water was around 2.5 NTU for 1000 ppm of [A336][CHB] used for demulsification. As the concentration of [A336][CHB] decreased from 1000 ppm to 300 ppm, the turbidity of separated water did not change significantly and stayed around 2.5 NTU.

The interfacial tension between toluene and deionized water was found to be ˜32 mN/m. When the toluene phase contained 300 ppm [A336][CHB], the interfacial tension between toluene and deionized water decreased to ˜10 mN/m, which was very close to the interfacial tension of [A336][C10]. Increasing the concentration of ionic liquids in toluene was found to lead to an increase in the interfacial tension. The presence of salt ions in water phase resulted in lower interfacial tension between water phase and toluene phase containing ionic liquids. The presence of salt ions in water lead to more stable interfacial tension over time.

Initial water content of water-in-bitumen emulsion was found to be an influential parameter affecting the demulsification efficiency of [A336][CHB]. For the water-in-bitumen emulsion containing ˜20% water, the demulsification efficiency reached ˜65%, using 100 ppm [A336][CHB] and after 3 hours of settling time. Increasing the settling time to 4-5 hours was found to increase the demulsification efficiency to ˜80%, which was lower than efficiency of its analogous fatty acid-based ionic liquid, [A336][C10].

For a water-in-bitumen emulsion with initial water content of ˜11%, the demulsification efficiency of 100 ppm [A336][CHB] reached ˜57% after 3 hours of settling time. Decreasing the concentration of ionic liquids to 50 ppm increased the demulsification efficiency to ˜78% after 4-5 hours of settling time.

For a water-in-bitumen emulsion with initial water content of ˜5-6%, the demulsification efficiency of 100 ppm [A336][CHB] reached ˜58% after 3 hours of settling time and it increased to ˜73% after 5 hours of settling time. It took ˜6 hours for [A336][CHB] to reduce the water content in the top oil phase to ˜1%. The demulsification efficiency of [A336][CHB] at low initial water content was similar or comparable to the efficiency of its analogous fatty acid based-ionic liquid, [A336][C10].

Decreasing the concentration of [A336][CHB] from 1000 ppm to 300 ppm did not significantly change the demulsification efficiency of [A336][CHB], but increasing the settling time from 3 hours to 4 hours was found to increase the demulsification efficiency of [A336][CHB] from ˜65% to ˜80%, when initial water content was ˜20% and ionic liquid concentration in the water-in-bitumen emulsion was 100 ppm.

The naphtha to bitumen (N/B) ratio had a significant effect on the demulsification efficiency of [A336][CHB]. Increasing the N/B ratio from 0.42 to 2 could increase the demulsification efficiency of 100 ppm [A336][CHB] to >99% in ˜2 hours and decreased the water content of the top oil phase to ˜300-500 ppm (0.03-0.05%).

### (iii) Example No. 3: Synthesizing Using Cyclohexyl Acetic Acid

Cyclohexyl acetic acid, as a cyclo-carboxylic acid with chemical formula of C₈H₁₃O₂, can also be used to prepare a second ionic liquid demulsifier based on a cyclo-carboxylic acid.

In this example, with reference to process (200) in FIG. 1—first, at step (202), trioctylmethylammonium hydroxide, [A336][OH], is synthesized by reacting a 0.5 M solution of trioctylmethylammonium chloride, [A336][Cl], in chloroform with a 5 M solution of sodium hydroxide in deionized water. The reaction can be carried out for one hour.

At step (204), the organic phase is decanted and equilibrated with a fresh 5 M NaOH solution. This procedure may be repeated for eight times and the chloride content is tested, for example, with an acidified silver nitrate solution to assure that the chloride content is negligible.

At step (206), the resulting organic layer is washed with deionized water and the solvent is. evaporated, at step (208), under vacuum to yield a viscous [A336][OH] liquid.

Importantly, at step (210), the [A336][OH] and cyclohexyl acetic acid are dissolved in dichloromethane (molar ratio 1:1) and refluxed at 30° C. for 12-14 hour. The solvent is evaporated (step (212)) using a rotary evaporator followed by thermal drying at 70° C. (step (214)) in a vacuum oven for 7-8 hours to yield trioctylmethylammonium 4-cyclohexylacetate, [A336][CHA]. The synthesis yield was ˜93%.

Viscosity of trioctylmethylammonium cyclohexyl acetate, [A336][CHA], at 23° C. was found to be 2.9 Pa.S. The viscosity of [A336][CHA] was higher than its analogous fatty acid-based ionic liquid, [A336][C8].

Thermal decomposition of [A336][CHA] started at ˜169° C. The thermal stability of [A336][CHA] and its analogous fatty acid-based ionic liquid, [A336][C8], were found to be almost similar to each other, however, full decomposition of [A336][C8] occurred at slightly higher temperature, while [A336][CHA] left a little residue on the thermogravimetric analysis pan.

Ionic liquid dosages ranging from 50 to 4000 ppm were studied for model water-in-oil emulsion separation to determine the optimum [A336][CHA] concentration range for actual demulsification process. The ionic liquid dosage stated hereafter is the total concentration of ionic liquid in whole volume of emulsion.

For model emulsion containing 800 mg/l of C5-asphaltenes and 20% deionized water, increasing the concentration of [A336][CHA] was found to lead to faster water separation, however, demulsification efficiency of low dosages (<600 ppm) was higher in longer settling times (>20 minutes). The water removal percentage of 98% was obtained by 100 ppm [A336][CHA] in 30 minutes and almost 100% of water is separated by 100 ppm and 50 ppm of [A336][CHA] in 60 minutes and 120 minutes, respectively.

Water content and demulsification efficiency of [A336][CHA] were found to have an inverse relation. As the water content decreased from 20% to 5%, the demulsification efficiency of 300 ppm [A336][CHA] decreased from 97% to 80%, after giving 2 hours of settling time.

For low water content emulsions, increasing the settling time favored the demulsification efficiency. While water separation occurred immediately (e.g., 5 minutes) when the water content of the emulsion was 20%, it took about 30-45 minutes for [A336][CHA] to see a considerable water separation when water content of the emulsion was as low as 5%.

The optimum temperature of demulsification for [A336][CHA] was realized around 60° C. to 80° C. The achieved results indicated that [A336][CHA] can be potentially used for demulsification of actual water-in-bitumen emulsion which is treated at ˜80° C. in industrial process.

The demulsification kinetics and efficiency of [A336][CHA] was also found to be higher than its analogous fatty acid-based ionic liquid, [A336][C8], at temperatures lower than 60° C.

The demulsification efficiency of 300 ppm [A336][CHA] after 120 minutes at 40° C., 60° C., and 80° C. was found to be 84%, 90%, and 99%, respectively.

Water salinity was found to lead to higher emulsion stability and lower water separation kinetics and efficiency in the case of model water-in-oil demulsification process. In the presence of salt (NaCl) ions in the emulsion, the water separation efficiency of [A336][CHA] was higher than its analogous fatty acid-based ionic liquid, [A336][C8]. Maximum demulsification efficiency of 300 ppm [A336][CHA] in the presence of 800 mg/l of NaCl ions was found to be 96%, which was obtained in 20 minutes.

The turbidity of separated water was around 1.1 NTU for 1000 ppm [A336][CHA] used for demulsification. As the concentration of [A336][CHA] decreased from 1000 ppm to 300 ppm, the turbidity of separated water decreased to 0.9 NTU.

The interfacial tension between toluene and deionized water was found to be ˜32 mN/m. When the toluene phase contained 300 ppm [A336][CHA], the interfacial tension between toluene and deionized water decreased to ˜7.5 mN/m, which was lower than the interfacial tension of its analogous fatty acid-based ionic liquid, [A336][C8], which was 9.5 mN/m. Increasing the concentration of ionic liquids in toluene lead to an increase in the interfacial tension. The presence of salt ions in water phase resulted in lower interfacial tension between water phase and toluene phase containing [A336][CHA]. The presence of salt ions in water lead to more stable interfacial tension over time.

Initial water content of water-in-bitumen emulsion was found to be an influential parameter affecting the demulsification efficiency of [A336][CHA]. For the water-in-bitumen emulsion containing ˜20% water, the demulsification efficiency reached ˜80%, using 100 ppm [A336][CHA] and after 3 hours of settling time. Increasing the settling time to 4-5 hours can increase the demulsification efficiency to ˜90-95%.

The demulsification efficiency of [A336][CHA] was also found to be slightly higher than the efficiency of its analogous fatty acid-based ionic liquid, [A336][C8], in ionic liquid concentrations above 300 ppm.

For a water-in-bitumen emulsion with initial water content of ˜11%, the demulsification efficiency of 100 ppm [A336][CHA] reached ˜68% after 3 hours of settling time. Decreasing the concentration of ionic liquids to 50 ppm increased the demulsification efficiency to ˜80-85% after 3-4 hours of settling time.

For a water-in-bitumen emulsion with initial water content of ˜5-6%, the demulsification efficiency of 100 ppm [A336][CHA] reached ˜55% after 3 hours of settling time and it increased to ˜76% after 5 hours of settling time. It took ˜6 hours for [A336][CHA] to reduce the water content in the top oil phase <1%.

Decreasing the concentration of [A336][CHA] from 1000 ppm to 100 ppm did not change the demulsification efficiency, but increasing the settling time from 3 hours to 4 hours was found to increase the demulsification efficiency of [A336][CHA] from ˜80% to ˜90%, when initial water content was ˜20% and ionic liquid concentration in the water-in-bitumen emulsion was 100 ppm.

The naphtha to bitumen (N/B) ratio was found to have a significant effect on the demulsification efficiency of [A336][CHA]. Increasing the N/B ratio from 0.42 to 2 was found to increase the demulsification efficiency of 100 ppm [A336][CHA] to >99% in ˜2 hours and decrease the water content of the top oil phase to ˜300-500 ppm (0.03-0.05%).

### (iv) Example No. 4: Synthesizing Using Naphthenic Acid

Naphthenic acid, as the third example of cyclo-carboxylic acid with chemical formula of C₁₀H₁₇O₂, can also be used to prepare an ionic liquid demulsifier based on a cyclo-carboxylic acid.

In this example, with reference to process (200) in FIG. 1—first, at step (202), trioctylmethylammonium hydroxide, [A336][OH], is synthesized by reacting a 0.5 M solution of trioctylmethylammonium chloride, [A336][Cl], in chloroform with a 5 M solution of sodium hydroxide in deionized water. The reaction can be carried out for one hour.

At step (204), the organic phase is decanted and equilibrated with a fresh 5 M NaOH solution. This procedure may be repeated for eight times and the chloride content is tested, for example, with an acidified silver nitrate solution to assure that the chloride content is negligible.

The resulting organic layer is then washed with deionized water (step (206)), and the solvent is evaporated under vacuum to yield a viscous [A336][OH] liquid (step (208)).

Importantly, at step (210), the [A336][OH] and naphthenic acid are dissolved in dichloromethane (molar ratio 1:1) and refluxed at 30° C. for 12-14 hours. The solvent is evaporated, at step (212), (e.g., using a rotary evaporator), followed by thermal drying at 70° C. in a vacuum oven for 7-8 hours to yield trioctylmethylammonium naphthenate, [A336][Nap] (step (214)). The synthesis yield was ˜94%.

Viscosity of trioctylmethylammonium naphthenate, [A336][Nap], at 23° C. was found to be 3.3 Pa.S. The viscosity of [A336][Nap] was 2.5 times the viscosity of its analogous fatty acid-based ionic liquid, [A336][C10]. Also, the viscosity of [A336][Nap] was slightly higher than its analogous cyclo-carboxylic acid-based ionic liquid with a cyclohexane in the carboxylate group.

Thermal decomposition of [A336][Nap] started at ˜160° C. The thermal stability of its analogous fatty acid-based ionic liquid, [A336][C10], was found to be higher. Full decomposition of [A336][Nap] occurred at 254° C., while leaving a little residue on the thermogravimetric analysis pan.

Results indicated that thermal stability of cyclo-carboxylate group containing cyclohexane was slightly higher than the thermal stability of cyclo-carboxylate group containing cyclopentane.

Ionic liquid dosages ranging from 50 to 4000 ppm were studied for model water-in-oil emulsion separation to determine the optimum [A336][Nap] concentration range for actual demulsification process. The ionic liquid dosage stated hereafter is the total concentration of ionic liquid in whole volume of emulsion.

For model emulsion containing 800 mg/l of C5-asphaltenes and 20% deionized water, demulsification kinetics was found to be high for all tested concentrations. Demulsification kinetics of higher concentrations was more, however, and the overall performance and maximum obtained water separation was higher for concentrations as low as 100 and 50 ppm. Water separation greater than 99% was found to be achieved in 20 and 30 minutes by 100 ppm and 50 ppm [A336][Nap], respectively.

Water content and demulsification kinetics of [A336][Nap] were found to have an inverse relation with each other. When the water content of the emulsion is 5%, ˜50% of water could be separated in 30 minutes with 300 ppm [A336][Nap], while ˜99% could be separated with the same [A336][Nap] concentration and settling time, when the water content was 20%.

For 5% water content, the water separation reached 91% after 2 hours of demulsification with 300 ppm [A336][Nap]. As the water content of the emulsion decreases, giving more time can increase the demulsification efficiency.

The optimum temperature of demulsification for [A336][Nap] was realized in a range of 60° C. to 80° C. The achieved results indicated that [A336][Nap] could be potentially used for demulsification of actual water-in-bitumen emulsion which was treated at ˜80° C. in industrial process.

The demulsification kinetics and efficiency of [A336][Nap] was higher than its analogous fatty acid-based ionic liquid, [A336][C10], as well as its analogous cyclo-carboxylic acid ionic liquid, [A336][CHA], at all temperatures. The trioctylmethyl ammonium naphthenate, [A336][Nap], was able to separate water at room temperature, as well.

Water salinity was found to lead to higher emulsion stability and lower water separation kinetics and efficiency in the case of model water-in-oil demulsification process. In the presence of salt (NaCl) ions, the water separation efficiency of [A336][Nap] was higher than its analogous fatty acid-based ionic liquid, [A336][C10]. In the presence of 800 mg/l NaCl ions, the maximum water separation of 93% was achieved with 300 ppm [A336][Nap] in 15 minute, while the water demulsification efficiency of 300 ppm [A336][Nap] in the absence of NaCl ions was ˜98% after 15 minutes of settling time.

The turbidity of separated water was ˜0.9 NTU for 1000 ppm [A336][Nap], and as the concentration of [A336][Nap] decreased from 1000 ppm to 300 ppm, the turbidity of separated water decreased to ˜0.4 NTU. The water turbidity of water phase separated with [A336][Nap] was lowest among all the example carboxylic acid-based ionic liquid demuslifiers.

The interfacial tension between toluene and deionized water was found to be ˜32 mN/m. When the toluene phase contained 300 ppm [A336][Nap], the interfacial tension between toluene and deionized water decreased to ˜6 mN/m. Increasing the concentration of ionic liquids in toluene lead to an increase in the interfacial tension. The presence of salt ions in water phase resulted in lower interfacial tension between water phase and toluene phase containing ionic liquids. The presence of salt ions in water lead to more stable interfacial tension over time.

Initial water content of water-in-bitumen emulsion was found to be an influential parameter affecting the demulsification efficiency of [A336][Nap]. For the water-in-bitumen emulsion containing ˜20% water, the demulsification efficiency reached ˜77%, using 100 ppm [A336][Nap] and after 3 hours of settling time. Increasing the settling time to 4-5 hours was found to increase the demulsification efficiency to ˜91%.

For a water-in-bitumen emulsion with initial water content of ˜11%, the dumulsification efficiency of 100 ppm [A336][Nap] reached ˜70% after 3 hours of settling time. Decreasing the concentration of ionic liquids to 50 ppm increased the demulsification efficiency to ˜88-92% after 5 hours of settling time.

For a water-in-bitumen emulsion with initial water content of ˜5-6%, the demulsification efficiency of 100 ppm [A336][Nap] reached ˜65% after 3 hours of settling time and it increased to ˜82% after 5 hours of settling time. It took ˜6 hours for [A336][Nap] to reduce the water content in the top oil phase to <1%.

Decreasing the concentration of [A336][Nap] from 1000 ppm to 100 ppm did not change the demulsification efficiency, but increased the settling time from 3 hours to 4 hours was found to increase the demulsification efficiency of [A336][Nap] from ˜78% to ˜88%, when the initial water content was ˜20% and the ionic liquid concentration in the water-in-bitumen emulsion was 100 ppm.

The demulsification efficiency of [A336][Nap] was higher than the efficiency of its analogous fatty acid-based ionic liquid, [A336][C10], in ionic liquid concentrations above 300 ppm.

The demulsification efficiency of [A336][Nap] was higher than the efficiency of its analogous cyclo-carboxylic acid-based ionic liquid, [A336][CHB], in the examined ionic liquid concentration and the initial water content ranges.

The naphtha to bitumen (N/B) ratio had a significant effect on the demulsification efficiency of [A336][Nap]. Increasing the N/B ratio from 0.42 to 2 increased the demulsification efficiency of 100 ppm [A336][Nap] to >99% in ˜2 hours and decreased the water content of the top oil phase to ˜300-500 ppm (0.03-0.05%).

### VII. INTERPRETATION

The description of the present invention has been presented for purposes of illustration and description, but it is not intended to be exhaustive or limited to the invention in the form disclosed. Many modifications and variations will be apparent to those of ordinary skill in the art without departing from the scope and spirit of the invention. Embodiments were chosen and described in order to best explain the principles of the invention and the practical application, and to enable others of ordinary skill in the art to understand the invention for various embodiments with various modifications as are suited to the particular use contemplated. To the extent that the following description is of a specific embodiment or a particular use of the invention, it is intended to be illustrative only, and not limiting of the claimed invention.

The corresponding structures, materials, acts, and equivalents of all means or steps plus function elements in the claims appended to this specification are intended to include any structure, material, or act for performing the function in combination with other claimed elements as specifically claimed.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to combine, affect or connect such aspect, feature, structure, or characteristic with other embodiments, whether or not such connection or combination is explicitly described. In other words, any element or feature may be combined with any other element or feature in different embodiments, unless there is an obvious or inherent incompatibility between the two, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an element, item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes the endpoints, each specific value, integer, decimal, fraction or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

1. A demulsifier compound of Formula I: 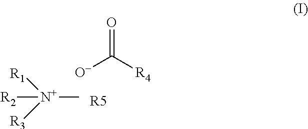
where R₁, R₂, and R₃ are, independently, branched or linear alkyl chains; R₄ is a saturated or unsaturated, linear, branched or cyclic moiety having between 7 to 17 carbon atoms; and R₅ is methyl, ethyl or propyl or isopropyl.

2. The compound of claim 1, wherein R₁, R₂, and R₃ each comprises a linear chain of 6 to 12 carbon atoms.

3. The compound of claim 1, wherein R₄ comprises an aliphatic or aromatic ring.

4. The compound of claim 3, wherein R₄ comprises cyclohexane or cyclopentane.

5. The compound of claim 1, which is a compound of Formula II: 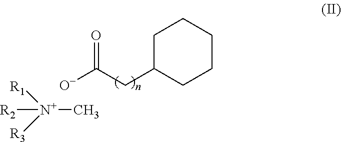
where n is 1 to 9.

6. The compound of claim 1, which is a compound of Formula III: 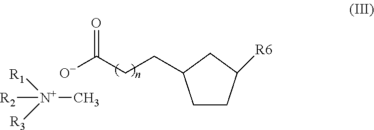
wherein R₆ is hydrogen, carbon or a linear or branched alkyl chain having between 2 and 13 carbon atoms; and n is between 0 and 12.

7. The compound of claim 6, wherein R₆ is methyl, ethyl, propyl or isopropyl.

8. The compound of claim 4, wherein the total number of carbon atoms of R₄ is less than or equal to 17.

9. A method of demulsifying a water-in-oil emulsion, comprising contacting the emulsion with a compound of claim 1.

10. The method of claim 9 which is conducted at between 40° C. and 90° C.

11. The method of claim 9 wherein the compound is present in a concentration of between about 50 ppm to about 4000 ppm.

12. The method of claim 11, wherein the concentration is about 1000 ppm.

13. The method of claim 9, wherein the demulsifying step is part of a hot water bitumen extraction process.

14. The method of claim 13, wherein the water content of the bitumen is between 1 to 25%.

15. The method of claim 14, wherein the demulsifying compounds of claims 1, 5, and 6 are used alone or in combination.

16. The method of claim 15, wherein the demulsifying compounds are dissolved in an organic solvent including naphtha, aromatic and aliphatic hydrocarbons, and alcohols.

17. The method of claim 15, wherein the demulsifying compounds are dissolved in a water/alcohol mixture.

18. The method of claim 15, wherein the demulsifying compounds are used at concentrations as low as 25 ppm.
